(19) United States
(12) Patent Application Publication
(10) Pub. No.: US 2025/0368589 A1
(43) Pub. Date: Dec. 4, 2025

(54) DUAL TOWER PROPANE RECOVERY PROCESS

(71) Applicant: Lummus Technology LLC, Houston, TX (US)

(72) Inventors: Galip Hakan Guvelioglu, Houston, TX (US); Fereidoun Yamin, Houston, TX (US)

(73) Assignee: Lummus Technology LLC, Houston, TX (US)

(21) Appl. No.: 19/226,043

(22) Filed: Jun. 2, 2025

Related U.S. Application Data
(60) Provisional application No. 63/654,807, filed on May 31, 2024.

(51) Int. Cl. C07C 7/09

(52) U.S. Cl. CPC C07C 7/09

(57) ABSTRACT

A process for recovering propane from a hydrocarbon stream including cooling a feed gas, separating the feed gas into a vapor and a liquid, expanding the vapor to produce a feed gas, feeding the feed gas to an absorber and recovering an absorber bottoms product, an absorber liquid draw, and an absorber overheads fraction; feeding the absorber bottoms product to a deethanizer and producing a deethanizer liquid fraction comprising propane and heavier components and a deethanizer overhead vapor fraction comprising ethane and lighter components. The absorber liquid draw, absorber overheads fraction, and deethanizer overhead vapor fraction are brought into indirect heat exchange to produce an absorber reboil stream and to partially condense the deethanizer overheads vapor product.

### FIELD OF THE DISCLOSURE

Embodiments herein relate to the recovery of hydrocarbons from natural gas and other light hydrocarbon containing gas streams.

### BACKGROUND

Various processes are utilized for the recovery of ethane, ethylene, propane, and propylene from a variety of gas streams. Some of the gas streams processed may include natural gas streams, refinery off gas streams, coal seam gas streams, and the like. In addition, these components may also be present in other sources of hydrocarbons such as coal, tar sands, and crude oil to name a few.

Various processes recover ethane, propane, and heavier components, such as U.S. Pat. Nos. 7,458,232, 7,484,385, 7,793,517, 7,818,979, 7,856,847, 5,568,737, 5,799,507, 4,689,063, 5,566,554, and 6,578,379, among others.

Various processes recover primarily propane and heavier components, rejecting ethane, such as U.S. Pat. Nos. 7,069,744, 6,712,880, 4,617,039, 5,771,712, 4,690,702, and 5,114,450, among others.

Many of these configurations for propane and ethane recovery are capital and energy intensive (high capital and operational cost).

### SUMMARY OF THE DISCLOSURE

Embodiments herein provide for systems and processes for the recovery of propane and heavier compounds from gas streams. More particularly, embodiments herein relate to the recovery of propane and heavier compounds from gas streams utilizing a simpler, lower capital and operationally intensive configuration that can achieve high (99+%) propane recovery with very low ethane recovery.

In one aspect, embodiments herein relate to a process for recovering propane from a hydrocarbon stream. The process includes feeding a gas stream comprising methane, ethane, propane, and heavier hydrocarbons to a feed heat exchanger; partially condensing the feed gas in the feed heat exchanger, producing a partially condensed feed gas; feeding the partially condensed feed gas to a cold separator, and recovering a feed gas fraction and a feed liquid fraction; and feeding the feed gas fraction as an absorber feed to an absorber and recovering an absorber bottoms product, an absorber liquid draw, and an absorber overheads fraction. At least a portion of the feed liquid fraction is fed to the feed heat exchanger to provide cooling in indirect heat exchange with the feed gas, and produce a heated liquid fraction. The absorber bottoms product and the heated liquid fraction are fed to a deethanizer for producing a deethanizer liquid fraction comprising propane and heavier components and a deethanizer overhead vapor fraction comprising ethane and lighter components. The absorber liquid draw is vaporized in indirect heat exchange with the deethanizer overheads vapor fraction (i) to produce an absorber reboil stream and (ii) to partially condense the deethanizer overheads vapor fraction to produce a partially cooled deethanizer overheads product, and (iii) the partially cooled deethanizer overheads product is cooled and condensed in indirect heat exchange with the absorber overhead fraction to recover a cooled deethanizer overheads stream and a warmed absorber overheads product. The cooled deethanizer overheads stream is fed as reflux to the absorber.

In another aspect, embodiments disclosed herein relate to a system for recovering propane from a hydrocarbon stream. The system includes a feed heat exchanger configured for receiving a feed gas comprising methane, ethane, propane, and heavier hydrocarbons and partially condensing the feed gas to produce a partially condensed feed gas. The partially condensed feed gas is fed to a cold separator, recovering a feed gas fraction and a feed liquid fraction. The feed gas fraction is fed to an absorber configured to produce an absorber bottoms product, an absorber liquid draw, and an absorber overheads fraction. A flow line is provided for feeding at least a portion of the feed liquid fraction to the feed heat exchanger to provide cooling in indirect heat exchange with the feed gas, and recovering a heated liquid fraction. The absorber bottoms product and heated liquid fraction are fed to a deethanizer configured for producing a deethanizer liquid fraction comprising propane and heavier components and a deethanizer overhead vapor product comprising ethane and lighter components. An absorber vaporizer is provided for vaporizing the absorber liquid draw via indirect heat exchange with the deethanizer overheads vapor product (i) to produce an absorber reboil stream, (ii) to partially condense the deethanizer overheads vapor product to produce a partially cooled deethanizer overheads, and (iii) further condensing and cooling the partially cooled deethanizer overheads via indirect heat exchange with the absorber overhead fraction to recover a cooled deethanizer overheads stream and a warmed absorber overheads product. A flow line is provided for feeding the cooled deethanizer overheads stream as reflux to the absorber.

Other aspects and advantages will be apparent from the following description and the appended claims.

### BRIEF DESCRIPTION OF DRAWINGS

FIG. 1 illustrates a simplified process flow diagrams for dual distillation tower systems for recovering propane according to embodiments herein.

FIG. 2 illustrates a simplified process flow diagrams for dual distillation tower systems for recovering propane according to embodiments herein.

FIG. 3 illustrates a simplified process flow diagrams for dual distillation tower systems for recovering propane according to embodiments herein.

FIG. 4 illustrates a simplified process flow diagrams for dual distillation tower systems for recovering propane according to embodiments herein.

FIG. 5 illustrates a simplified process flow diagrams for dual distillation tower systems for recovering propane according to embodiments herein.

FIG. 6 illustrates a simplified process flow diagrams for dual distillation tower systems for recovering propane according to embodiments herein.

FIG. 7 illustrates a simplified process flow diagrams for dual distillation tower systems for recovering propane according to embodiments herein.

FIG. 8 illustrates a simplified process flow diagrams for dual distillation tower systems for recovering propane according to embodiments herein.

### DETAILED DESCRIPTION

Processes and systems herein relate to the recovery of propane from gas mixtures, such as natural gas mixtures. Inlet gases (feeds) that may be processed according to embodiments herein may include methane, C2 compounds (ethane, ethylene), C3 compounds (propane, propylene), and hydrocarbons having 4 or greater carbon atoms (i.e., compounds heavier than C3 compounds). The term “C3+ compounds” means all organic compounds having three or more carbon atoms, including aliphatic species such as alkanes, olefins, and alkynes, and, in particular, propane, propylene, methyl-acetylene and the like. The terms “C2+ compounds” and “C4+ compounds are similarly defined, respectively, for mixtures including 2 or more, or 4 or more, carbon atoms. Conversely, “C2−” refers to mixtures including 2 or fewer carbon atoms.

Feeds to embodiments herein may also include carbon dioxide, nitrogen, and other trace gases. Other impurities that may be present include water, hydrogen sulfide, and other compounds that may be detrimental to processing at low or cryogenic temperatures. As such, raw feed gases containing such impurities may be treated to remove such impurities, and the feed gas may be dried and filtered before being sent for propane recovery according to embodiments herein.

The inlet gas is initially cooled, resulting in a two-phase stream. The two-phase stream is then separated in a cold separator (e.g., a flash drum or other single stage vapor/liquid separation device). In some embodiments, all liquids from the cold separator are cooled by Joule-Thompson expansion by depressurization across a valve, and the resulting two-phase stream is used to cool the feed gas, and then the resulting lower pressure, warmed two-phase stream is sent to a deethanizer as a feed to an intermediate tray of the column (mid-feed).

The vapor from the cold separator is sent to a turboexpander where it is expanded and then the expander outlet two-phase colder stream is fed to a reboiled absorber tower. In some embodiments, in addition to, or instead of the turboexpander, an expansion valve may be disposed between the cold separator and the reboiled absorber tower. The absorber tower has two sections, a stripping section and a rectifying section. Rectifying is achieved by utilization of the deethanizer condenser overhead, which is further cooled and condensed using the absorber overhead and fed as reflux for the absorber in the rectification section. The absorber overhead is the residue/sales gas, which is warmed against the deethanizer condenser overhead and feed gas and compressed via a booster compressor and residue compressor.

The reboiled absorber includes a reboiler that warms a side or bottom draw from the absorber column, using heat from the deethanizer overhead stream. Due to the heat exchange, the deethanizer overhead stream may be condensed, as noted above, and fed as reflux to the absorber. In other words, the absorber reflux condenser also provides heat to the absorber, acting also as an absorber reboiler, and these terms may be used interchangeably herein (absorber reflux condenser and absorber reboiler). Further, the vapor from the reboiler provides heat to the stripping section of the absorber.

The absorber and deethanizer are operated at temperatures and pressures which are suitable for the desired separation of propane from gas mixtures, such as natural gas mixtures. Such temperatures and pressures are known in the art.

The reboiled absorber overhead vapor stream contains a majority of the ethane and lighter components. The bottom liquid recovered from the absorber contains some methane, ethane, propane and heavier components. The absorber bottoms liquid is fed to the deethanizer for further separation of the methane and ethane, all of the methane and the majority of ethane become part of the deethanizer column overhead stream, and a majority of the propane is recovered in the liquid bottom stream from the deethanizer column. The deethanizer condenser may be of a simple design which utilizes conventional propane refrigeration for lower capital expense (low CAPEX) and ease of operation.

In some embodiments, a portion of the liquid from the cold separator is sent to an absorber as an option.

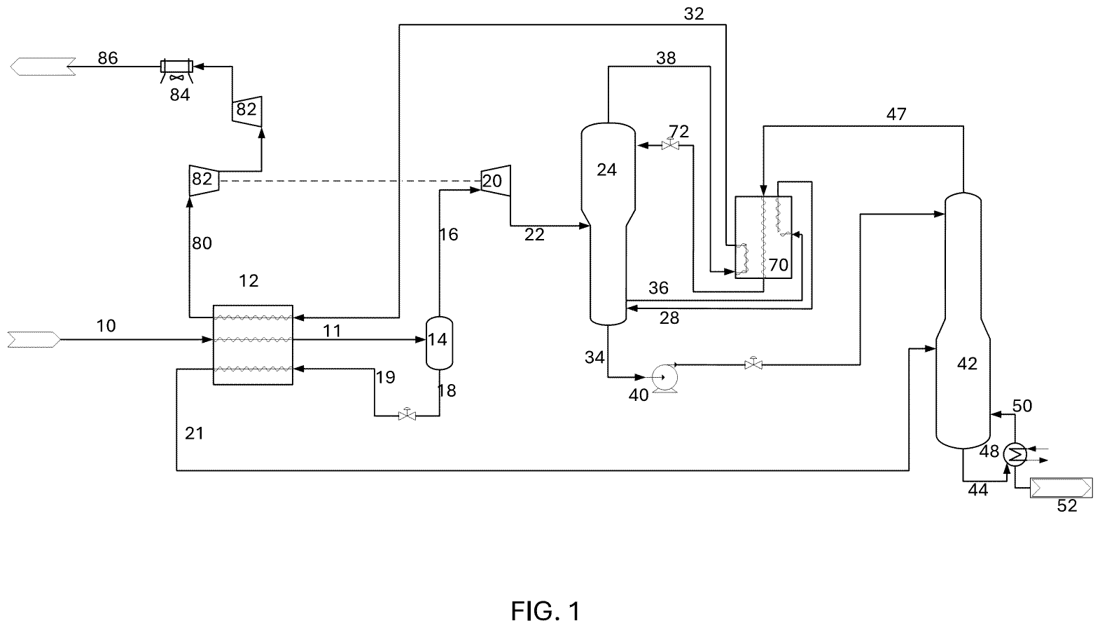

Referring now to FIG. 1, a simplified process flow diagram of a dual tower system for recovering propane according to embodiments is illustrated. A feed gas 10, including methane, ethane, propane, and optionally heavier hydrocarbons, is initially cooled in feed heat exchanger 12, partially condensing the feed gas. The cooled and partially condensed feed gas 11 is fed to cold separator 14, and the two phases are separated into a feed gas 16 and a feed liquid 18.

Cooling in the feed heat exchanger is provided by cross-exchange of the feed with the feed liquid 18 recovered from the cold separator 14 and which may be expanded across valve 19 upstream of feed heat exchanger 12. Cooling of the feed in feed heat exchanger 12 is also provided by cross-exchange with absorber overhead stream 32.

Feed gas 16 is expanded in expander 20, producing an expanded and cooled feed gas 22, which is fed to reboiled absorber 24. Feeds to the reboiled absorber include the expanded and cooled feed gas 22, reboiled vapor stream 28, and absorber reflux 72. Products recovered from the reboiled absorber 24 include absorber bottoms draw 34, absorber reboil draw 36, and absorber overhead draw 38. As noted above, feed gas 22 may be fed to the reboiled absorber as a mid-feed, such as at a tray intermediate the rectifying and stripping sections of the reboiled absorber.

Absorber reboil draw 36 (or absorber liquid draw) may be withdrawn from a sump of the reboiled absorber in some embodiments. In other embodiments, absorber reboil draw 36 may be withdrawn from a lower tray of the reboiled absorber. The reboiled vapor stream 28 adds heat to the absorber column and enhances the separation of ethane from the feed gas 22. The effect of the reboiler in removing ethane and lighter components from the feed gas lightens the load that would be placed on the deethanizer for removing the additional ethane that would otherwise be recovered with the absorber bottoms 34.

Absorber bottoms 34 is pumped, such as via an absorber bottoms pump 40, and fed to the deethanizer 42. Expanded feed liquid 21 is also fed to the deethanizer. Typically, the absorber bottoms 34 is fed to a tray in an upper portion of the deethanizer, or may be fed to a top tray of the deethanizer, while the expanded feed liquid 21 is fed to a tray in the lower portion of the deethanizer. In the deethanizer 42, the propane and heavier components are separated from the ethane and lighter components. C3+ hydrocarbons are recovered as a bottoms draw 44 from the deethanizer, while C2− hydrocarbons are recovered as a deethanizer overhead draw 47 from the deethanizer.

Bottoms draw 44 may be fed to a reboiler 48, such as a kettle reboiler or other reboiler sufficient for heating and separating C2 and C3 components, vaporizing a portion of the bottoms draw. The reboiled vapors 50 may be returned to the deethanizer 42, providing heat for the desired separations, and the remainder of the bottoms draw may be recovered as a liquid propane product stream 52.

Deethanizer overhead draw 47 may be cooled and condensed in the absorber reflux condenser 70, and fed to the reboiled absorber 24 as a reflux stream 72. Cooling of the deethanizer overhead draw 47 is provided by heat exchange with absorber overhead draw 38 and with absorber reboil draw 36.

After being heated against the deethanizer overhead draw 47, the warmed absorber overhead stream 32 is fed to feed heat exchanger 12 and used to cool the incoming inlet gas 10. The resulting C2− product stream 80 may then be compressed in one or more compression stages 82, then cooled using an aftercooler 84 to the desired C2− product temperature. The compression and cooling may be suitable to provide a residue product (C2− product) 86 having a temperature and pressure appropriate for feed of the C2− product into a natural gas pipeline.

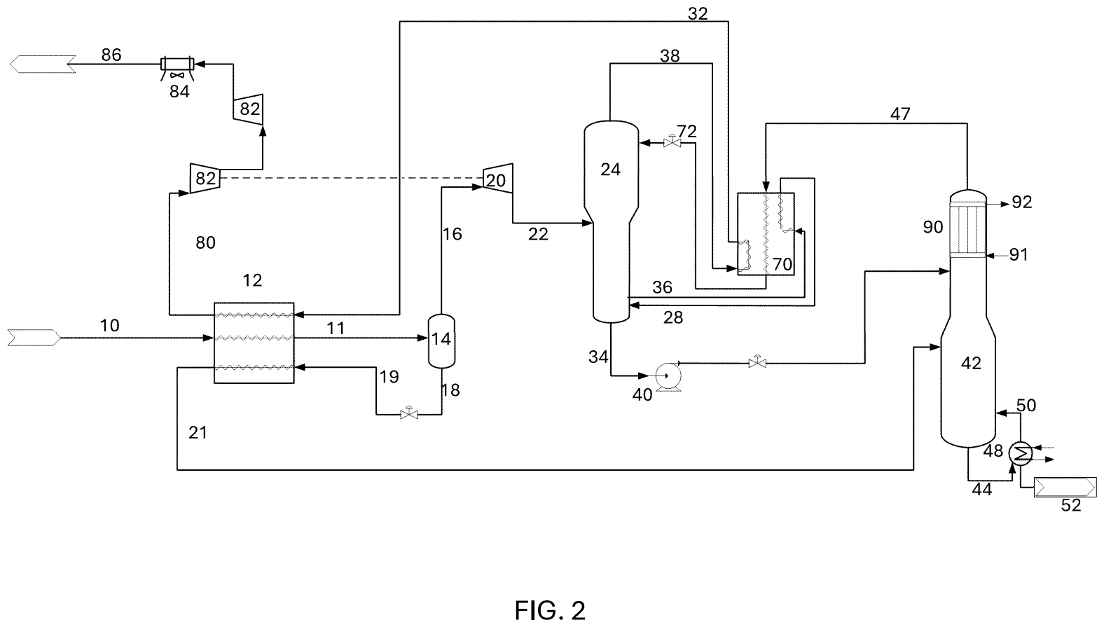

Referring now to FIG. 2, a simplified process flow diagram of a dual tower system for recovering propane according to embodiments is illustrated, where like numerals represent like parts as illustrated in FIG. 1 and previously described. In FIG. 2, the deethanizer 42 is equipped with an internal deethanizer condenser 90. Such an internal deethanizer condenser may be a knockback type or dephlegmator type condenser. Partially condensing of the vaporous material within the deethanizer 42 may be achieved in the internal deethanizer condenser 90 by feeding the internal deethanizer condenser 90 with a cold refrigerant 91. The cold refrigerant 91 chills and partially condenses the vapors at the top of the column to produce internal reflux and is recovered as a warmed refrigerant 92. The remaining (uncondensed) vapors are withdrawn from deethanizer 42 and recovered as the deethanizer overhead draw 47. In some embodiments (not illustrated), instead of a cold refrigerant 91, the deethanizer condenser 90 may be fed with the warmed absorber overhead stream 32, which is at a sufficiently low temperature to chill and partially condense the vapors at the top of the column. The warmed absorber overhead stream recovered from the deethanizer condenser is then fed to feed heat exchanger 12. The remaining portions of the process proceed as previously described.

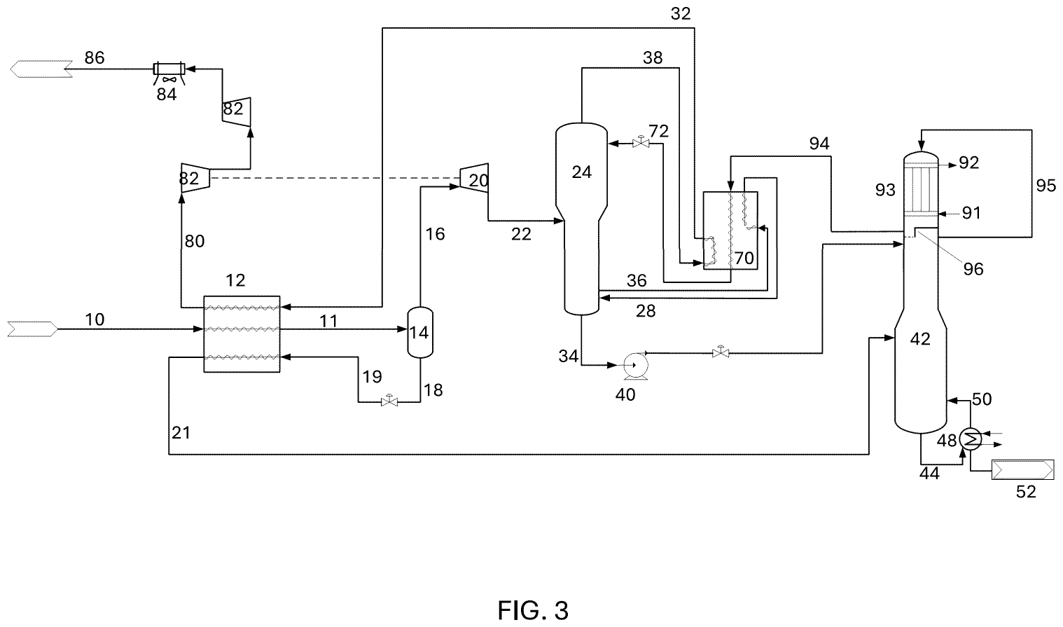

Referring now to FIG. 3, a simplified process flow diagram of a dual tower system for recovering propane according to embodiments is illustrated, where like numerals represent like parts as illustrated in FIGS. 1 and 2 and previously described. In FIG. 3, the deethanizer 42 is equipped with a vapor draw tray 96 and a downflow integrated condenser 93. The absorber bottoms 34 is pumped, such as via an absorber bottoms pump 40, and fed to the deethanizer 42 below the vapor draw tray 96. A vaporous fraction 95 is recovered from a region below the vapor draw tray 96 and fed to the top of the deethanizer 42. The \vaporous fraction then traverses downward through the downflow integrated condenser 93 and is cooled and partially condensed. The liquid condensate collects on vapor draw tray 96 and initiates liquid traffic at the top of the deethanizer column. The non-condensed vapors from downflow integrated condenser 93 are recovered via flow line 94 and fed to the absorber reflux condenser 70. In some embodiments (not illustrated), instead of a cold refrigerant 91, the deethanizer condenser 90 may be fed with the warmed absorber overhead stream 32, which is at a sufficiently low temperature to chill and partially condense the vapors at the top of the column. The warmed absorber overhead stream recovered from the deethanizer condenser is then fed to feed heat exchanger 12. The remaining portions of the process proceed as previously described.

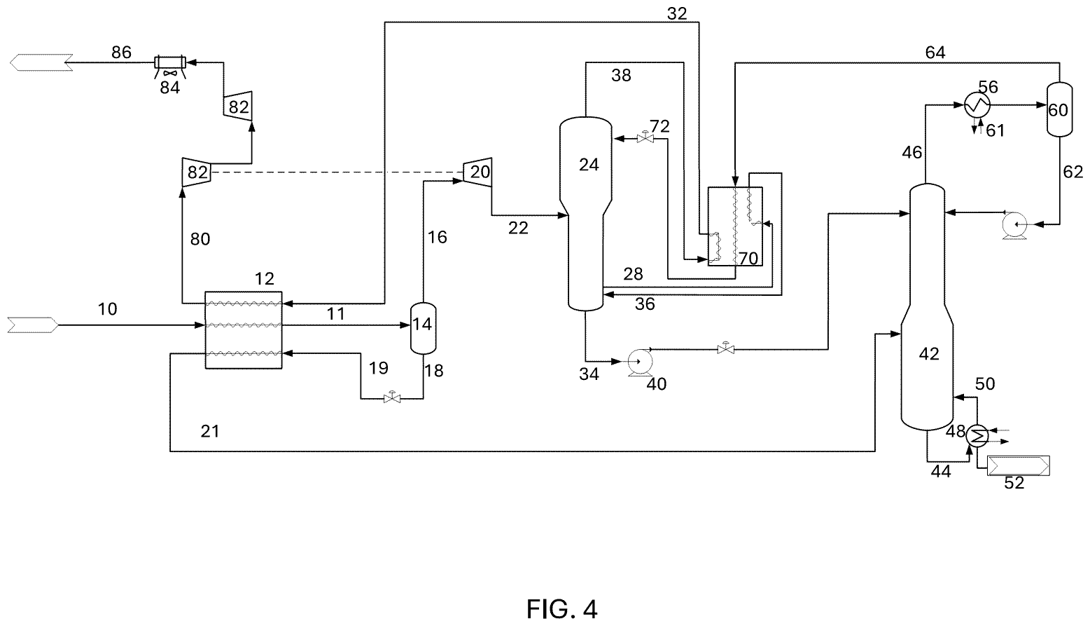

Referring now to FIG. 4, a simplified process flow diagram of a dual tower system for recovering propane according to embodiments is illustrated. A feed gas 10, including methane, ethane, propane, and optionally heavier hydrocarbons, is initially cooled in feed heat exchanger 12, partially condensing the feed gas. The cooled and partially condensed feed gas 11 is fed to cold separator 14, and the two phases are separated into a feed gas 16 and a feed liquid 18.

Cooling in the feed heat exchanger is provided by cross-exchange of the feed with the feed liquid 18 recovered from the cold separator 14 and which may be expanded across valve 19 upstream of feed heat exchanger 12. Cooling of the feed in feed heat exchanger 12 is also provided by cross-exchange with absorber overhead stream 32.

Feed gas 16 is expanded in expander 20, producing an expanded and cooled feed gas 22, which is fed to reboiled absorber 24. Feeds to the reboiled absorber include the expanded and cooled feed gas 22, reboiled vapor stream 28, and absorber reflux 72. Products recovered from the reboiled absorber 24 include absorber bottoms draw 34, absorber reboil draw 36, and absorber overhead draw 38. As noted above, feed gas 22 may be fed to the reboiled absorber as a mid-feed, such as at a tray intermediate to the rectifying and stripping sections of the reboiled absorber.

Absorber reboil draw 36 may be withdrawn from a sump of the reboiled absorber in some embodiments. In other embodiments, absorber reboil draw 36 may be withdrawn from a lower tray of the reboiled absorber. The reboiled vapor stream 28 adds heat to the absorber column and enhances the separation of ethane from the feed gas 22. The effect of the reboiler in removing ethane and lighter components from the feed gas lightens the load that would be placed on the deethanizer for removing the additional ethane that would otherwise be recovered with the absorber bottoms 34.

Absorber bottoms 34 is pumped, such as via an absorber bottoms pump 40, and fed to the deethanizer 42. Expanded feed liquid 21 is also fed to the deethanizer. Typically, the absorber bottoms 34 is fed to a tray in an upper portion of the deethanizer, while the expanded feed liquid 21 is fed to a tray in the lower portion of the deethanizer. In the deethanizer 42, the propane and heavier components are separated from the ethane and lighter components. C3+ hydrocarbons are recovered as a bottoms draw 44 from the deethanizer, while C2− hydrocarbons are recovered as an overhead draw 46 from the deethanizer.

Bottoms draw 44 may be fed to a reboiler 48, such as a kettle reboiler or other reboiler sufficient for heating and separating C2 and C3 components, vaporizing a portion of the bottoms draw. The reboiled vapors 50 may be returned to the deethanizer 42, providing heat for the desired separations, and the remainder of the bottoms draw may be recovered as a liquid propane product stream 52.

Deethanizer overhead draw 46 may be partially condensed in deethanizer condenser 56 and the resulting vapor and liquid may be separated in deethanizer reflux drum 60. Cooling for the deethanizer condenser may be provided, for example, using cold refrigeration 61, which may be from an open loop or closed loop refrigeration system. The resulting condensed liquids 62 may be fed as a reflux to the deethanizer. The remaining overhead vapor stream 64 may be recovered from deethanizer reflux drum 60, cooled and condensed in the absorber reflux condenser 70, and fed to the reboiled absorber as a reflux stream 72. Cooling of the deethanizer overhead product stream 64 is provided by heat exchange with absorber overhead draw 38 and with absorber reboil draw 36.

In some embodiments (not illustrated), instead of a cold refrigerant 61, the deethanizer condenser 56 may be fed with the warmed absorber overhead stream 32, which is at a sufficiently low temperature to chill and partially condense the deethanizer overhead draw. The warmed absorber overhead stream recovered from the deethanizer condenser is then fed to feed heat exchanger 12.

After being heated against the deethanizer reflux drum overhead product stream 64, the warmed absorber overhead stream 32 is fed to feed heat exchanger 12 and used to cool the incoming inlet gas. The resulting C2− product stream 80 may then be compressed in one or more compression stages 82, then cooled using an aftercooler 84 to the desired C2− product temperature. The compression and cooling may be suitable to provide a residue product (C2− product) 86 having a temperature and pressure appropriate for feed of the C2− product into a natural gas pipeline.

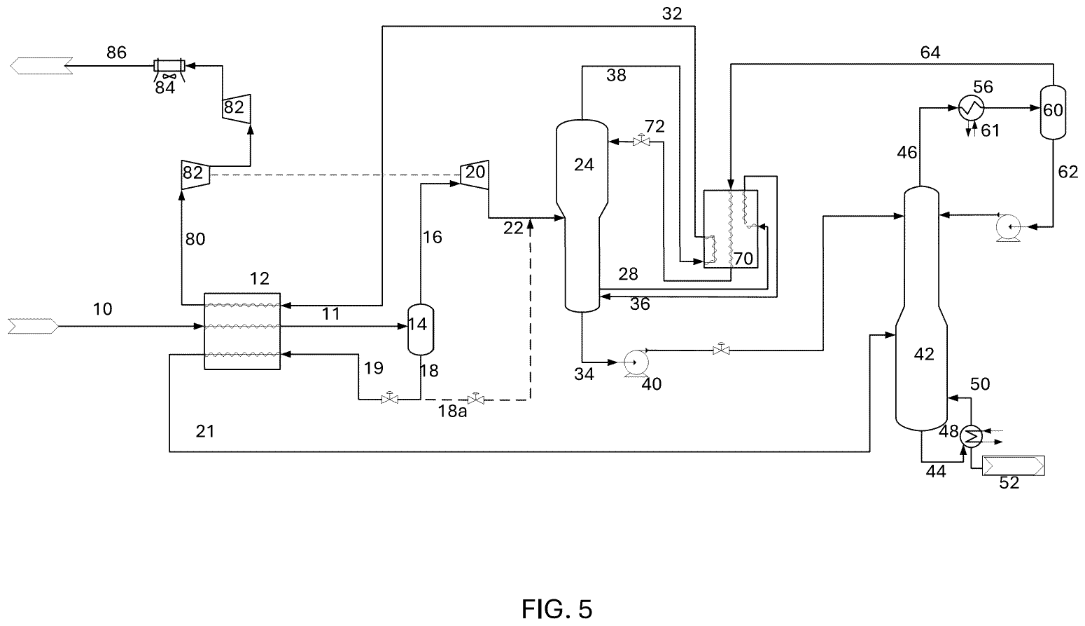

Referring now to FIG. 5, a simplified process flow diagram of a dual tower system for recovering propane according to embodiments is illustrated, where like numerals represent like parts as illustrated in FIG. 4 and previously described. Inlet gas 10 is processed in a manner similar to that as described for FIG. 4, however in this embodiment, a portion 18a of the liquid feed 18 recovered from cold separator 14 is fed to reboiled absorber 24 together with the feed gas 22.

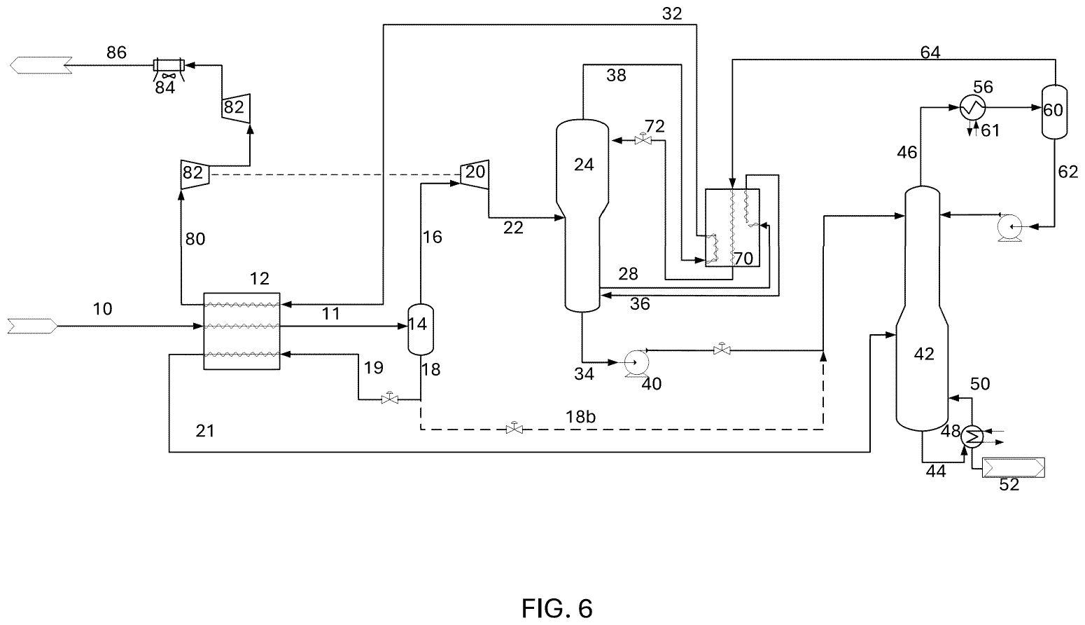

Referring now to FIG. 6, a simplified process flow diagram of a dual tower system for recovering propane according to embodiments is illustrated, where like numerals represent like parts as illustrated in FIGS. 4 and 5 and previously described. Inlet gas 10 is processed in a manner similar to that as described for FIGS. 4 and 5, however in this embodiment, a portion 18b of the liquid feed 18 recovered from cold separator 14 is combined with the absorber bottoms liquid and fed to the deethanizer 42.

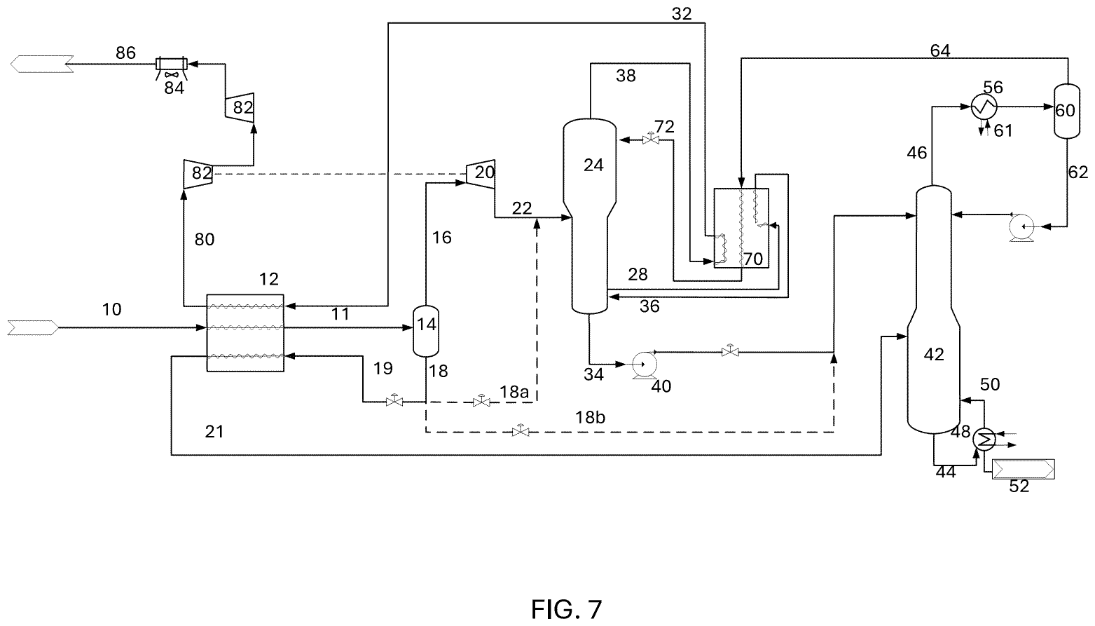

Referring now to FIG. 7, a simplified process flow diagram of a dual tower system for recovering propane according to embodiments is illustrated, where like numerals represent like parts as illustrated in FIGS. 1-6 and previously described. Inlet gas 10 is processed in a manner similar to that as described for FIGS. 1-5, however in this embodiment, a portion 18a of the liquid feed 18 recovered from cold separator 14 is fed to reboiled absorber 24 together with the feed gas 22 and a portion 18b of the liquid feed 18 recovered from cold separator 14 is combined with the absorber bottoms liquid and fed to the deethanizer 42.

FIGS. 5-7, presenting various feed flow embodiments, are only illustrated with respect to the flow scheme of FIG. 4, having an overhead condenser associated with the deethanizer. While not illustrated, it should be understood that the feed variations described in FIGS. 5-7 are also contemplated herein as being applicable to the embodiments of FIGS. 1-3 (deethanizer variations).

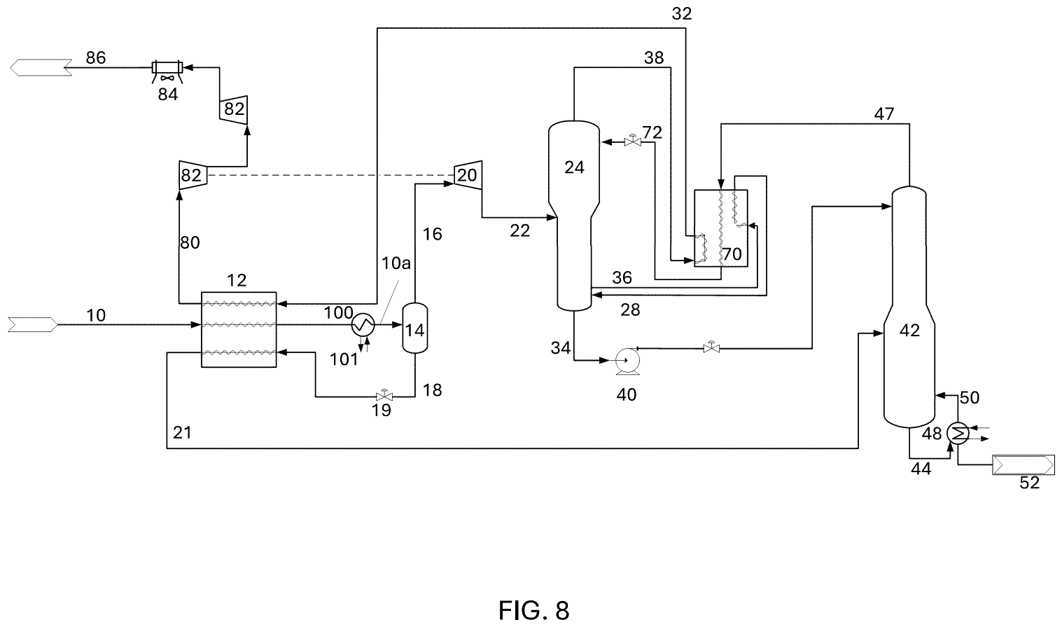

Referring now to FIG. 8, a simplified process flow diagram of a dual tower system for recovering propane according to embodiments is illustrated, where like numerals represent like parts as illustrated in FIGS. 1-7 and previously described. The partially condensed feed gas exiting the feed heat exchanger 12 is further cooled and condensed in a pre-separator condenser 100. Cooling for the pre-separator condenser 100 may be provided, for example, using propane refrigeration 101, which may be from an open loop or closed loop refrigeration system. The resulting stream 10a is then fed to the cold separator 14 and the remaining portions of the process proceed as previously described.

While only illustrated with respect to the embodiments on FIG. 1, it should be understood that the additional feed heat exchanger of the embodiment described in FIG. 8 are also contemplated herein as being applicable to the embodiments of FIGS. 1-7 (feed flow variations and deethanizer variations).

Embodiments herein further elucidate the ability to use all or a portion of the residue gas 38 to provide additional cooling to one or both of the feed heat exchanger 12 and the absorber reflux condenser 70.

As described above, embodiments herein provide for a simple yet efficient manner for recovering propane from an inlet gas. Embodiments herein advantageously utilize a reboiled absorber to allow for enhanced and efficient separation of propane from the inlet gas. For example, embodiments herein may provide for efficient propane recovery while requiring 15 to 20% less compression power as compared to the processes in U.S. Pat. No. 7,069,744.

Embodiments herein also provide for a simple yet efficient manner for recovering propane and propylene from an inlet gas which includes an olefin mixture. In such embodiments, propylene may be recovered with propane in the deethanizer bottoms product while ethylene is recovered with ethane in the deethanizer overheads product.

While the disclosure includes a limited number of embodiments, those skilled in the art, having benefit of this disclosure, will appreciate that other embodiments may be devised which do not depart from the scope of the present disclosure. Accordingly, the scope should be limited only by the attached claims.

What is claimed:

1. A process for recovering propane from a hydrocarbon stream, the process comprising:
feeding a feed gas comprising methane, ethane, propane, and heavier hydrocarbons to a feed heat exchanger;
partially condensing the feed gas in the feed heat exchanger, producing a partially condensed feed stream;
feeding the partially condensed feed stream to a cold separator, and recovering a feed gas fraction and a feed liquid fraction;
feeding the feed gas fraction as an absorber feed to an absorber and recovering an absorber bottoms product, an absorber liquid draw, and an absorber overheads fraction;
feeding at least a portion of the feed liquid fraction to the feed heat exchanger, providing cooling in indirect heat exchange with the feed gas, producing a heated liquid fraction;
feeding the absorber bottoms product and the heated liquid fraction to a deethanizer and producing a deethanizer liquid fraction comprising propane and heavier components and a deethanizer overhead vapor product comprising ethane and lighter components;
partially vaporizing the absorber liquid draw via indirect heat exchange with the deethanizer overheads vapor product (i) to produce an absorber reboil stream and (ii) to partially condense the deethanizer overhead vapor product to produce a partially cooled deethanizer overheads;
further condensing and cooling the partially cooled deethanizer overheads via indirect heat exchange with the absorber overheads fraction to recover a cooled deethanizer overheads stream and a warmed absorber overheads product; and
feeding the cooled deethanizer overheads stream as reflux to the absorber.

2. The process of claim 1, further comprising partially condensing a vaporous fraction in the deethanizer using an internal deethanizer condenser, recovering non-condensed vapors from the internal deethanizer condenser as the deethanizer overheads vapor product.

3. The process of claim 2, further comprising:
feeding an external refrigerant fluid to the internal deethanizer condenser to partially condense the vaporous fraction; or
feeding the warmed absorber overheads product to the internal deethanizer condenser to partially condense the vaporous fraction; or
feeding the external refrigerant fluid and the warmed absorber overheads product to the internal deethanizer condenser to partially condense the vaporous fraction.

4. The process of claim 1, further comprising expanding the feed gas fraction, producing an expanded feed gas and feeding the expanded feed gas to the absorber as the absorber feed.

5. The process of claim 1, further comprising recovering a deethanizer overheads vapor fraction from the deethanizer, partially condensing the deethanizer overheads vapor fraction in a deethanizer condenser, recovering a partially condensed deethanizer overheads product and feeding the partially condensed deethanizer overheads product to a deethanizer reflux drum to recover the deethanizer overheads vapor product and a deethanizer overheads liquid reflux.

6. The process of claim 5, further comprising:
feeding an external refrigerant fluid to the deethanizer condenser to partially condense the deethanizer overheads vapor fraction; or
feeding the warmed absorber overheads product to the deethanizer condenser to partially condense the deethanizer overheads vapor fraction; or
feeding the external refrigerant fluid the warmed absorber overheads product to the deethanizer condenser to partially condense the deethanizer overheads vapor fraction.

7. The process of claim 5, further comprising recycling the deethanizer overheads liquid reflux to the deethanizer.

8. The process of claim 1, further comprising feeding the heated liquid fraction to the deethanizer.

9. The process of claim 1, further comprising feeding the warmed absorber overheads product to the feed heat exchanger to provide further cooling to the feed gas.

10. The process of claim 1, further comprising feeding a remaining portion of the feed liquid fraction to the absorber.

11. The process of claim 1, further comprising feeding a remaining portion of the feed liquid fraction to the deethanizer.

12. The process of claim 1, further comprising feeding a first portion of a remaining portion of the feed liquid fraction to the absorber and a second portion of the remaining portion of the feed liquid fraction to the deethanizer.

13. The process of claim 1, further comprising further cooling and partially condensing the partially condensed feed stream in a pre-separator partial condenser, wherein an external refrigerant fluid is fed to the pre-separator condenser to facilitate the further cooling and partially condensing.

14. The process of claim 1, wherein the feed further comprises a mixture of olefins, and the deethanizer liquid fraction comprises propane, propylene, and heavier components and the deethanizer overhead vapor product comprised ethane, ethylene and lighter components.

15. The process of claim 1, further comprising:
recovering a vaporous fraction from the deethanizer as a side draw;
feeding the vaporous fraction to a top of the deethanizer, and partially condensing the vaporous fraction in the deethanizer using a downflow integrated condenser, producing a partially condensed deethanizer product; and
separating non-condensed vapors from condensate in the partially condensed deethanizer product, and withdrawing the non-condensed vapors as the deethanizer overheads vapor product.

16. The process of claim 15, further comprising:
feeding an external refrigerant fluid to the downflow integrated condenser to partially condense the vaporous fraction; or
feeding the warmed absorber overheads product to the downflow integrated condenser to partially condense the vaporous fraction; or
feeding the external refrigerant fluid and the warmed absorber overheads product to the downflow integrated condenser to partially condense the vaporous fraction.

17. A system for recovering propane from a hydrocarbon stream, the system comprising:
a feed heat exchanger configured for receiving a feed gas comprising methane, ethane, propane, and heavier hydrocarbons and partially condensing the feed gas to produce a partially condensed feed gas;
a cold separator configured for receiving the partially condensed feed gas and producing a feed gas fraction and a feed liquid fraction;
an absorber configured for receiving the feed gas fraction and producing an absorber bottoms product, an absorber liquid draw, and an absorber overheads fraction;
a feed line configured for feeding at least a portion of the feed liquid fraction to the feed heat exchanger after a pressure let down via Joule-Thompson expansion across a valve resulting in a two-phase stream that provides cooling via indirect heat exchange with the feed gas and producing a heated two-phase stream;
a deethanizer configured for receiving the absorber bottoms product and at least a portion of the heated two-phase stream and producing a deethanizer liquid fraction comprising propane and heavier components and a deethanizer overhead vapor product comprising ethane and lighter components;
an absorber vaporizer configured for vaporizing the absorber liquid draw via indirect heat exchange with the deethanizer overheads vapor product (i) to produce an absorber reboil stream, (ii) to partially condense the deethanizer overheads vapor product to produce a partially cooled deethanizer overheads, and (iii) further condensing and cooling the partially cooled deethanizer overheads via indirect heat exchange with the absorber overheads fraction to recover a cooled deethanizer overheads stream and a warmed absorber overheads product; and
a flow line configured for feeding the cooled deethanizer overheads stream as reflux to the absorber.

18. The system of claim 17, further comprising a feed gas expander, and expansion valve, or both, disposed between the cold separator and the absorber and configured for expanding the feed gas fraction to produce an expanded feed gas, wherein the expanded feed gas is fed to the absorber.

19. The system of claim 17, further comprising:
a deethanizer condenser configured for partially condensing the deethanizer overheads vapor fraction and recovering a partially condensed deethanizer overheads product; and
a deethanizer reflux drum configured for receiving the partially condensed deethanizer overheads product and producing a deethanizer overheads vapor product and a deethanizer overheads liquid reflux.

20. The system of claim 19, further comprising:
(i) an external refrigerant fluid system configured for providing an external refrigerant to the deethanizer condenser to partially condense the deethanizer overheads product; or
(ii) a flow line configured for feeding the warmed absorber overheads product to the deethanizer condenser to partially condense the deethanizer overheads product; or
a combination of (i) and (ii).

21. The system of claim 17, further comprising a feed line configured for feeding a remaining portion of the feed liquid fraction to the absorber with the feed gas fraction.

22. The system of claim 17, further comprising a feed line configured for feeding a remaining portion of the feed liquid fraction to the deethanizer with the absorber bottoms product.

23. The system of claim 17, further comprising a first feed line configured for feeding a first portion of a remaining portion of the feed liquid fraction to the absorber with the feed gas fraction and a second feed line configured for a second portion of the remaining portion of the feed liquid fraction to the deethanizer with the absorber bottoms product.

24. The system of claim 17, further comprising an internal deethanizer condenser disposed within an upper portion of the deethanizer, the internal deethanizer condenser configured for partially condensing a vaporous fraction and producing a deethanizer overheads product, and a flow line configured for feeding the deethanizer overheads product to the absorber reflux condenser as the deethanizer overheads vapor product.

25. The system of claim 24, further comprising:
(i) an external refrigerant fluid system configured for providing an external refrigerant to the internal deethanizer condenser to partially condense the vaporous fraction; or
(ii) a flow line configured for feeding the warmed absorber overheads product to the internal deethanizer condenser to partially condense the vaporous fraction; or
a combination of (i) and (ii).

26. The system of claim 17, further comprising a pre-separator condenser disposed between the feed heat exchanger and the cold separator and configured for further cooling and partially condensing the partially condensed feed gas, wherein an external refrigerant fluid is fed to the pre-separator condenser to facilitate the further cooling and partially condensing.

27. The system of claim 17, further comprising:
a side draw from the deethanizer configured for recovering a vaporous fraction;
a downflow integrated condenser disposed in an upper portion of the deethanizer and configured for receiving the vaporous fraction at a top of the deethanizer, and partially condensing the vaporous fraction, producing a partially condensed deethanizer product; and
a vapor outlet configured for separating non-condensed vapors from condensate in the partially condensed deethanizer product, and producing the non-condensed vapors as the deethanizer overheads vapor product.

28. The system of claim 27, further comprising:
(i) an external refrigerant fluid system configured for providing an external refrigerant to the downflow integrated condenser to partially condense the vaporous fraction; or
(ii) a flow line configured for feeding the warmed absorber overheads product to the downflow integrated condenser to partially condense the vaporous fraction; or
a combination of (i) and (ii).
